(19) United States
(12) Patent Application Publication
(10) Pub. No.: US 2003/0104432 A1
(43) Pub. Date: Jun. 5, 2003

(54) Methods of amplifying sense strand RNA

(75) Inventors: Zhidong Xu, San Francisco, CA (US); David Jablons, San Francisco, CA (US); Liang You, San Francisco, CA (US); Biao He, Millbrae, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/206,613

(22) Filed: Jul. 26, 2002

Related U.S. Application Data
(60) Provisional application No. 60/308,190, filed on Jul. 27, 2001.

(51) Int. Cl. C12Q 1/68; C12P 19/34

(52) U.S. Cl. 435/6; 435/91.2

(57) ABSTRACT

The present invention provides efficient and novel methods for synthesizing and amplifying sense-strand RNA. The methods of the invention include methods of synthesizing probes useful for probing oligo and cDNA microarrays and for the development of subtractive and normalized expression libraries.

### CROSS-REFERENCES TO RELATED APPLICATIONS

This application is claims priority to provisional application serial No. 60/308,190 filed on Jul. 26, 2001, herein incorporated by reference in its entirety.

### STATEMENT AS TO RIGHTS TO INVENTIONS MADE UNDER FEDERALLY SPONSORED RESEARCH OR DEVELOPMENT

This invention was made with Government support under National Cancer Institute grant number R21 CA85172-01. The Government has certain rights in the invention.

### BACKGROUND OF THE INVENTION

The completion of the entire human genome sequence has provided huge amounts of DNA sequence information for biomedical research. One of the most remarkable applications of the human genome information is microarray technology. Using high-density arrays of oligo nucleotides or complementary DNA, a large number of gene expression profiles have been generated from cellular activities involved in disease, cancer, cell cycles, environmental exposure, and many other biological events.

Micro-arrays can be made from cDNA, genomic DNA, and most recently, oligo nucleotide primers. See, e.g., Lockhart et al., Nature 405:827-836 (2000). Previously, fragments of DNA were spotted on the nylon membrane and hybridized with radiolabeled probes for screening differentially expressed genes. Recently, glass slides have been used as a substrate for arrayed DNA spots and more powerful detection systems have been used with fluorescence for detection. Recent studies comparing cDNA-based and oligo primer-based arrays for gene expression profiling suggest that oligo nucleotide based microarray provide greater specificity in target sequence design and detection.

At least three methods have been used for preparing labeled materials for measurement of gene expression. For example, the RNA can be labeled directly using photo-biotinylation. Alternatively, the labeled nucleotides can be incorporated into cDNA during a reverse transcriptase reaction or labeled nucleotides can be incorporated into antisense RNA (“aRNA”) through in vitro transcription. See, e.g., Van Gelder et al., Proc. Natl. Acad. Sci. USA 87:1663-1667 (1990). In the last case, a T7 promotor is incorporated by reverse transcriptase reaction into cDNA. The double stranded cDNA then serves as template for in vitro transcription using T7 RNA polymerase, during which labeled nucleotides are incorporated into aRNA. See, e.g., Van Gelder et al., supra.

A large hurdle in this area of research is that relatively large amounts of RNA is required for preparing labeled materials for array analysis, ranging from a few micrograms of mRNA to grater than 50 μg of total RNA. See, e.g., Wang et al., Nature Biotech. 18:457-459 (2000). In addition, the need for large amounts of RNA limits microarray hybridization analysis from microdissected samples, such as LCM (laser capture microscopy), small tumor specimens and subcellular samples, such as neuronal cells, cell sorting samples, and early developmental organs where only minute amounts of RNA is available.

Presently available technologies for producing RNA expression sequences from a small amount of starting material amplify antisense RNA and produce a transcription template by incorporating a promoter primer to the polyA tail (3′ end) of messenger RNA or the 5′ end of the first strand cDNA. This approach has limitations on the quality of RNA that can be obtained. The problems with transcribing from a transcription promoter incorporated to the polyA tail are that a substantial number of RNA sequences are not represented in their full length or simply not represented at all, due to 5′ exon sequences that do not get transcribed, in part because of intron splicing that occurs before synthesis of the first strand of cDNA, and the fact that 3′ untranslated regions are highly variable in length, from a few hundred base pairs to a few thousand base pairs. The result is a population of expressed sequences that are biased to the 3′ ends of the mRNA and a final population pool of expressed sequences that is not representative of the original population pool. Furthermore, because most commercially available synthetic oligonucleotide microarrays are designed to contain coding sequences of genes, use of antisense mRNA amplified from the 3′ end messages in such microarrays is likely not to fully inform of expressed sequences. The final result is likely not representative of the relative abundance of individual mRNA sequences within an RNA population. The present invention addresses these and other problems.

### BRIEF SUMMARY OF THE INVENTION

The present invention provides methods of generating long sense strand RNA (lsRNA). In some aspects, the methods of the invention comprise: providing a first strand cDNA comprising a 5′ and a 3′ end; incorporating a promoter primer comprising a promoter regulatory element onto the 3′ of the first strand cDNA; synthesizing a second strand cDNA complementary to the first strand cDNA, thereby incorporating the promoter primer into a double-stranded (ds) cDNA; and initiating transcription of the cDNA, thereby generating a long sense strand RNA.

In one embodiment, incorporating the promoter primer is ligating said promoter primer. In one embodiment, incorporating the promoter primer is by reverse transcription.

In one embodiment, the first strand cDNA is synthesized from a first sense strand RNA sequence isolated from a biological sample. In a further embodiment the biological sample comprises submicrogram quantities of total RNA.

In some embodiments, the promoter regulatory element is from a promoter selected from the group consisting of T7, T3 and SP6 promoters. In some aspects, the method further comprises the steps of synthesizing a first strand cDNA from the sense strand RNA and repeating the steps of the methods described above, i.e., ligating a promoter primer comprising a promoter regulatory element onto the 3′ of the first strand cDNA; synthesizing a second strand cDNA complementary to the first strand cDNA, thereby incorporating the promoter primer into a double-stranded cDNA; and initiating transcription of the cDNA, thereby generating a sense strand RNA.

In one embodiment, the second strand of cDNA is synthesized by PCR amplification. In another embodiment, the second strand of cDNA is synthesized by primer extension.

In a preferred embodiment the generated long sense strand RNA is a transcript of a full length RNA sequence.

In some aspects, the promoter primer is biotin labeled. In some aspects, the double-stranded cDNA is purified with magnetic beads. In some aspects, the transcription of the cDNA occurs when the cDNA is anchored to the magnetic beads. In some aspects, the magnetic beads are linked to streptavidin.

In some aspects, the first strand cDNA comprises a poly dT sequence.

In some aspects, the promoter primer comprises a T7 regulatory element. In some aspects, the promoter primer comprises a T3 regulatory element. In some aspects, the promoter primer comprises a SP6 regulatory element.

In some aspects, the promoter primer is single stranded. In some aspects, the promoter is ligated to the first strand cDNA by a T4 RNA ligase. In some aspects, the single stranded promoter primer is phosphorylated in the 5′ end.

In some aspects, the promoter primer is double stranded. In some aspects, the promoter primer is ligated to the first strand cDNA by a T4 DNA ligase. In some aspects, the promoter primer comprises an overhanging single stranded sequence at least partially complementary to the first strand cDNA. In some aspects, the promoter primer comprises random nucleotides on the 3′ end of the primer, i.e. the 3′ end of the polynucleotide sequence that hybridizes to the first strand cDNA. In some aspects, the promoter primer comprises 6-10 random nucleotides on the 3′ end of the primer.

In some aspects, the methods of the invention further comprise amplification of the double stranded cDNA. In some embodiments, the transcription comprises incorporation of labeled nucleotides into the sense strand RNA, thereby synthesizing a labeled sense strand RNA. In some aspects, the labeled nucleotides are fluorescent nucleotides. In some aspects, the method further comprises probing a polynucleotide array with the labeled sense strand RNA.

In some aspects, the methods further comprise reverse transcribing the sense strand RNA, thereby synthesizing a single-stranded (ss) cDNA probe. In some aspects, the reverse transcription step is performed in the presence of labeled nucleotides, thereby synthesizing a labeled single-stranded cDNA probe. In some aspects, the nucleotides are labeled with fluorescent dye. In some aspects, the fluorescent dye is selected from the group consisting of cy3 and cy5.

In one embodiment, the generated sense strand RNA is directly hybridized to a nucleic acid microarray that comprises complementary polynucleotides.

The invention also provides methods for generating sense strand RNA using single-strand cDNA as a template.

In one method, this method comprises the steps of: isolating an RNA sequence from a population pool of RNA sequences from a biological sample; synthesizing a first strand of cDNA in a reverse transcription reaction and including in the cDNA synthesis reaction (i) an oligo dT primer, (ii) a promoter primer comprising a promoter regulatory element and a first 3′ nucleotide overhang sequence, and (iii) a second nucleotide overhang sequence that is complementary to the first nucleotide overhang sequence, thereby inducing the reverse transcriptase to switch templates from the isolated RNA sequence to the promoter primer, thereby making a double-stranded promoter primer and a RNA/cDNA duplex.

In one embodiment, the first nucleotide overhang sequence and the second nucleotide sequence is a trinucleotide. In a further embodiment the trinucleotide is GGG or CCC.

In one embodiment, the method comprises the further steps of digesting the isolated RNA sequence with a RNase, initiating in vitro transcription of the single-stranded cDNA from the double-stranded promoter primer, thereby generating sense strand mRNA using single-strand cDNA as a template.

In another embodiment, the method comprises the further steps of concurrently providing the mRNA/cDNA duplex with a RNase, a DNA polymerase and a ligase, thereby synthesizing a second strand of cDNA complementary to the first strand of cDNA; and initiating transcription of the cDNA, thereby generating sense strand mRNA.

In another method, long sense strand RNA is generated from a single-stranded cDNA template in a method comprising the steps of: isolating an RNA sequence from a population pool of RNA sequences from a biological sample; synthesizing a first strand of cDNA in a reverse transcription reaction; ligating a double-stranded promoter primer sequence to the 3′ end of the first strand of cDNA, thereby generating a single-stranded cDNA sequence with a double-stranded promoter sequence at the 3′ end; and initiating transcription of the cDNA, thereby generating sense strand RNA using single-strand cDNA as a template.

The invention also provides for a method of generating long antisense strand RNA, the method comprising the steps of: providing a first strand cDNA using a downstream primer comprised of oligo dT and a first promoter; incorporating an upstream promoter primer comprised of a second promoter to the 3′ end to said first strand cDNA; amplifying double-stranded cDNA by PCR; and generating long antisense RNA by in vitro transcription with the polymerase for said first promoter, whereby said long antisense strand RNA is generated.

The invention also provides methods of generating a mixture of sense strand of mRNAs. In some aspects, the methods comprise: providing a pool of first strand cDNAs comprising a 5′ and a 3′ end; incorporating a promoter primer comprising a T7, T3 or SP6 promoter onto the 3′ of the first strand cDNAs; synthesizing second strand cDNAs complementary to the first strand cDNAs, thereby incorporating the promoter primer into a double-stranded cDNAs; and initiating transcription of the double-stranded cDNAs, thereby generating a mixture of sense strand mRNAs.

In one embodiment, incorporating the promoter primer is ligating said promoter primer. In one embodiment, incorporating the promoter primer is by reverse transcription.

In some aspects, the method further comprises normalizing a cDNA library with the mixture of sense strand mRNAs. In some aspects, the mixture of sense strand mRNAs are biotinylated and the method further comprises the steps of contacting in a solution the mixture of sense strand mRNAs with the cDNA library, thereby forming mRNA/cDNA hybrids; and separating the hybrids from solution.

In another aspect, the invention provides a method of generating sense strand mRNA from a biological sample, such as a tissue sample, comprising partially degraded mRNA.

In one embodiment, the steps of generating sense strand mRNA includes the steps of isolating partially degraded mRNA from a biological sample; ligating a polyA tail to the 3′ end of the isolated mRNA; synthesizing a first strand of cDNA by reverse transcription reaction; ligating a promoter primer comprising a promoter regulatory element onto the 3′ of the first strand cDNA; synthesizing a second strand of cDNA complementary to the first strand of cDNA, thereby incorporating the promoter primer into a double-stranded cDNA; and amplifying sense mRNA by in vitro transcription, thereby generating a sense strand mRNA.

In one embodiment, the second strand of cDNA is synthesized by PCR amplification. In another embodiment, the second strand of cDNA is synthesized by primer extension.

In one embodiment, the step of generating sense strand mRNA includes the steps of isolating partially degraded mRNA from a first tissue; generating short cDNA fragments by a reverse transcription reaction; isolating full length mRNA sequences from a cellular source of a second tissue that is the same tissue type as the first tissue; extending the short cDNA fragments by a second reverse transcription reaction, using the full length mRNA as a template; ligating a promoter primer comprising a promoter regulatory element onto the 3′ of the extended cDNA; and amplifying sense mRNA by in vitro transcription, thereby generating sense strand mRNA.

In a preferred embodiment, the amplified sense strand mRNA is a population of full length mRNA sequences.

In some embodiments, the in vitro transcription step is performed in the presence of labeled nucleotides, thereby synthesizing a labeled single-stranded cDNA probe. In some aspects, the nucleotides are labeled with fluorescent dye. In some aspects, the fluorescent dye is selected from the group consisting of cy3 and cy5.

In one embodiment, the method includes the step of directly hybridizing the sense mRNA to a microarray slide.

In one embodiment the sense RNA is used in RT-PCR analysis. In one embodiment, the sense strand mRNA is used as a nucleic acid probe in a hybridization reaction. In another embodiment, the sense strand mRNA is used to construct a cDNA library.

In one embodiment, the biological sample having partially degraded mRNA is a paraffin-embedded tissue sample.

The invention also provides for a kit for use in amplifying long sense strand RNA. A kit of the invention comprises: (i) enzymes for carrying out reactions for amplification of sense strand mRNA, including a reverse transcriptase, a DNA polymerase, a RNA polymerase, and a ligase, such as a DNA ligase or a RNA ligase; (ii) upstream and downstream primers, wherein said upstream primer is comprised of a double-stranded promoter primer sequence comprising a 3′ overhang, and wherein said downstream primer is comprised of an oligo dT sequence; and (iii) instructions for use of the kit to generate long sense strand RNA.

In one embodiment, the promoter of the promoter primer sequence is selected from the group consisting of T7, T3 and SP6. 1431 In one embodiment, the 3′ overhang is comprised of random nucleotides. In one embodiment, the 3′ prime overhang is comprised of 6-10 random nucleotides.

### Definitions

“First strand cDNA” refers to a single-stranded DNA molecule that is complementary to an RNA molecule, preferably a messenger RNA molecule.

A “cDNA” is a single-stranded or double-stranded DNA molecule that complements a RNA molecule, preferably a messenger RNA (mRNA) molecule. A “full-length cDNA” is a single or double-stranded DNA molecule that contains the complete sequence of a RNA molecule. In particular, a full length cDNA contains substantially the complete 3′ and 5′ sequences of a RNA molecule. A “substantially complete” cDNA sequence is preferably at least 90% identical, more preferably at least 95% identical and most preferably at least 99% identical a mRNA molecule.

As used herein, a “cDNA library” refers to a population of single-stranded or double-stranded DNA molecules that have the sequence, or the complementary sequence, of a population of mRNA molecules. Typically, the mRNA molecules serve as a template for the construction of the cDNA molecules.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19: 5081; Ohtsuka et al. (1985) J. Biol. Chem. 260: 2605-2608; Cassol et al. (1992); Rossolini et al. (1994) Mol. Cell. Probes 8: 91-98). The term nucleic acid is used interchangeably with gene, cDNA, and mRNA encoded by a gene.

The term “promoter” or “promoter regulatory element” refers to a region or sequence located upstream and/or downstream from the start of transcription and which are involved in recognition and binding of RNA polymerase and other proteins to initiate transcription. “Inducible promoter” refers to a promoter that directs expression of a gene where the level of expression is alterable by environmental or developmental factors such as, for example, temperature, pH, transcription factors and chemicals.

A DNA segment is “operably linked” when placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof.

A “primer” refers to a single or double stranded nucleic acid sequence. Typically, the primer comprises fewer than 200 nucleotides, and more preferably fewer than 50 nucleotides. A “promoter primer” refers to a primer that comprises a promoter regulatory element.

The term “isolated”, when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. One of skill will recognize that polynucleotides can be manipulated in many ways and are not limited to the examples above.

### BRIEF DESCRIPTION OF THE DRAWINGS

FIG. 1 illustrates a flow chart for the amplification of sense RNA.

FIG. 2 illustrates some aspects for developing constructs useful for sense RNA amplification.

FIG. 3 illustrates a modified procedure for sense RNA amplification on solid phase support.

FIG. 4 illustrates a method for adding a T7 promoter anchor sequence to 3′ ends of first strand cDNA using T4 RNA ligase.

FIG. 5 illustrates a variation of the method depicted in FIG. 3 that employs T4 RNA ligase to ligate a T7 promoter primer to the 3′ ends of the first strand cDNA.

FIG. 6 illustrates a “template switching mechanism” for sense RNA amplification using a double-stranded promoter and a single-stranded template.

FIG. 7 illustrates a strategy for sense RNA amplification with in vitro transcription using a double-stranded promoter and a single-stranded template.

FIG. 8 illustrates an application of sense RNA in microarray hybridization. Amplified sense strand mRNA is reverse transcribed in the presence of labeled nucleotides to synthesize labeled antisense cDNA that can be directly hybridized to a double-stranded cDNA microarray or a single-stranded oligonucleotide microarray.

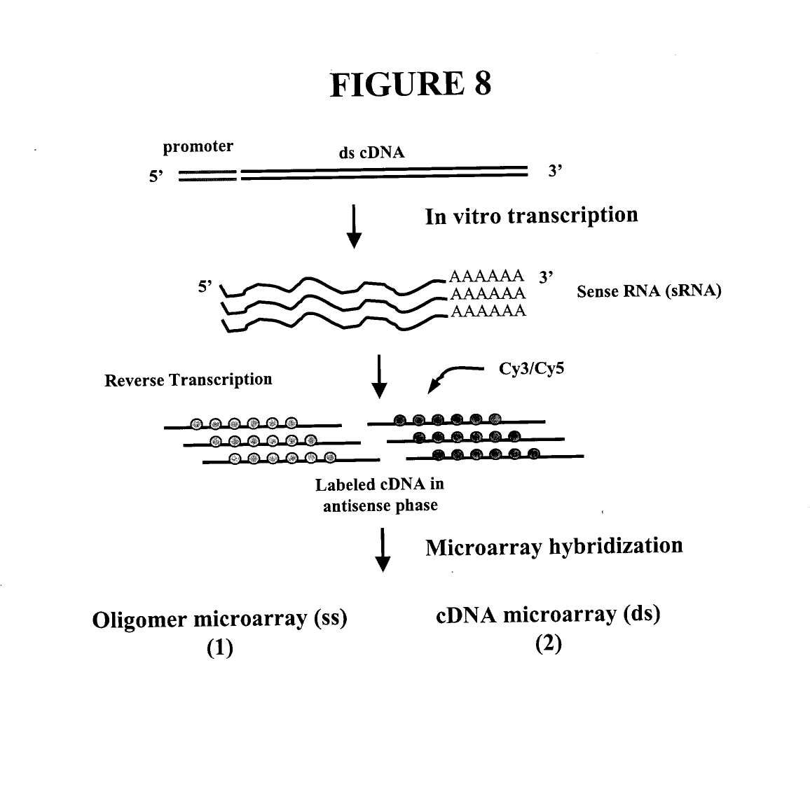

FIG. 9 illustrates gene expression profiling by microarray analysis using sense RNA amplified from RNA samples taken from paraffin blocks.

FIG. 10 demonstrates the achievement of microgram quantity yields of sense RNA by PCR amplification of the cDNA template and then using T7 in vitro transcription.

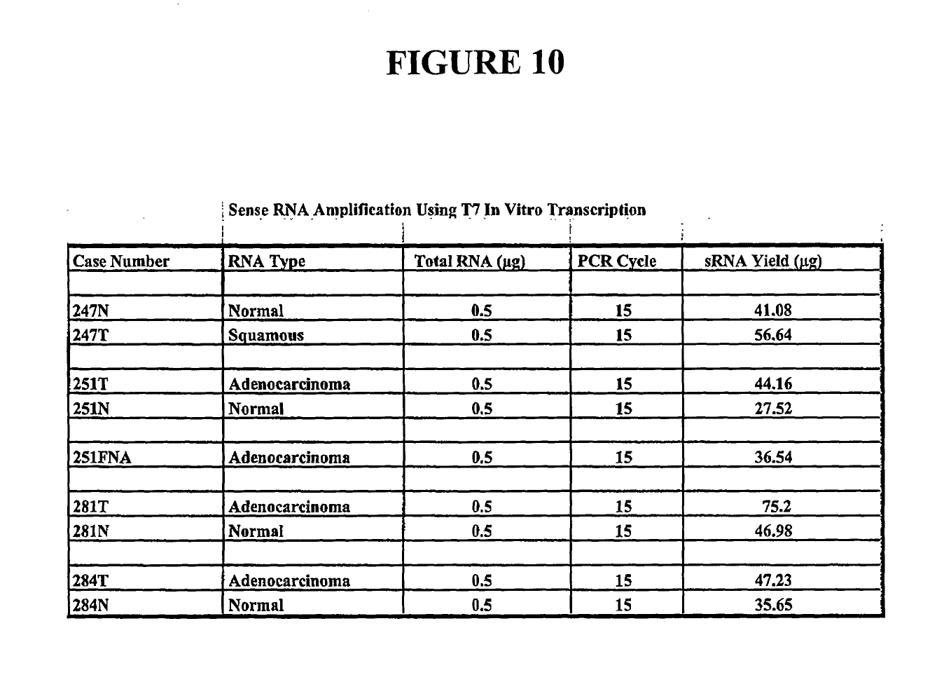

FIG. 11 demonstrates the achievement of microgram quantity yields of sense RNA by primer extension of the cDNA template and then using T7 in vitro transcription.

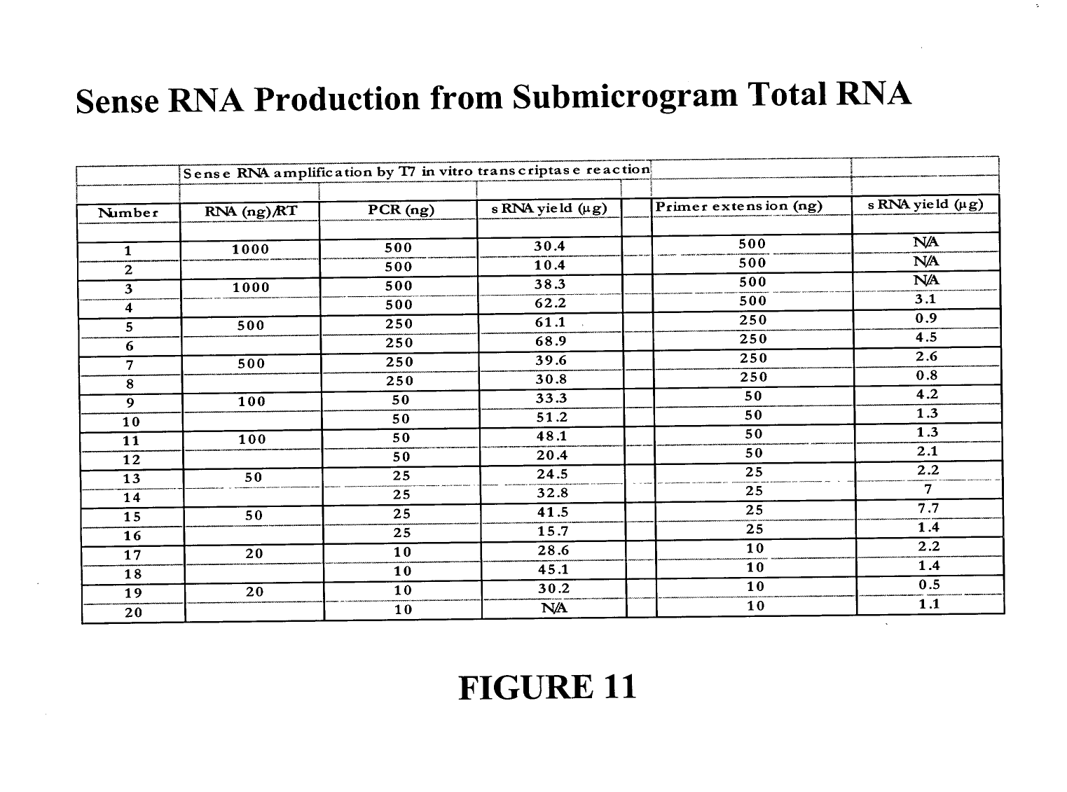

FIG. 12 illustrates a comparison of methods for in vitro RNA amplification.

FIG. 13 illustrates a method of generating long antisense RNA.

### DETAILED DESCRIPTION OF THE INVENTION

I. Introduction

The present invention relates to methods for amplification of full-length sense RNA (sRNA) by in vitro transcription. The methods comprise ligating a promoter primer to the 3′ ends of first strand cDNAs (corresponding to the 5′ ends of messenger RNAs (mRNA)). In one method, ligating a double stranded promoter primer to the 3′ end of a first strand cDNA generates a single stranded cDNA template with a double stranded promoter that can be directly used for the generation of sense strand RNA by in vitro transcription. In another method, a second cDNA strand is synthesized (e.g., by primer extension using a promoter specific sense primer or by PCR amplification under limited cycle numbers using a promoter-specific sense primer). The double-stranded cDNAs containing a promoter sequence downstream of the 3′ ends of the antisense template strand of cDNA molecules are then used for sense RNA synthesis and amplification using an in vitro transcription system.

The methods of the present invention are distinct over currently available technologies, which generate antisense mRNA and incorporates a promoter sequence to the 5′ end of the first strand of cDNA. It was a surprisingly found that driving sense RNA amplification by preparing an antisense cDNA template with a promoter primer incorporated onto its 3′ end allows for the more efficient capture of full length and rare mRNA sequences, thereby providing a higher quality mRNA population pool of longer sequence lengths that is more representative of the mRNA expression profile from the original biological sample. Additionally, the present methods for generating long strand RNA is efficient. For instance, the synthesis of a second strand of cDNA can be eliminated. This allows for the determination of an expression profile of a biological sample in considerably less time than what is required using currently available technologies. The present methods are capable of amplifying large amounts of sense RNA from submicrogram quantities of starting total RNA materials. Large quantities of sense RNA are particularly useful, e.g., for DNA microarray (gene chip) hybridization and for preparing driver molecules for normalization/subtraction cDNA libraries.

II. General Recombinant DNA Methods

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al, eds., 1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides can be performed by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981). 1721 A method for isolating specific cDNA molecules combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences directly from mRNA, from cDNA, from genomic libraries or cDNA libraries. Degenerate oligonucleotides can be designed to amplify gene homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of specific mRNA in physiological samples, for nucleic acid sequencing, or for other purposes. Genes amplified by the PCR reaction can be purified from agarose gels and cloned into an appropriate vector.

III. First Strand cDNAs

Methods for the construction of first strand cDNA molecules are well known in the art. See, e.g., Current Protocols in Molecular Biology (Ausubel et al., eds., 1994). In general, high quality mRNA molecules from a source of interest is used as a template for a reverse transcription reaction. Any reverse transcriptase can be used. For instance, RNaseH⁻, thermal stable or regular reverse transcriptase can be used.

The present invention was designed to use small amount of RNA as template. For example, quantities ranging from 1 μg to 10 ng of RNA can be used for first strand cDNA synthesis. In some embodiments, less than 1 μg of RNA, and preferably less than about 50 ng of RNA and more preferably less than about 20 ng of RNA is used as starting RNA.

Depending of the strategy employed for cDNA cloning, numerous cDNA synthesis primers can be used for the first-strand synthesis. The primer can be a single stranded oligonucleotide, a double-stranded oligonucleotide with a single-stranded portion (see, e.g., Coleclough, et al. Gene 34:305-14 (1985)) or a vector primer, representing a double stranded vector with a partly single stranded portion (Okayma, et al., Mol. Cell Bio. 2:161-170 (1982)). For full length cDNA library construction, it is preferred that the primer contain an oligo dT tail at the 3′ end. This oligo dT region binds to the mRNA poly A tail, thus beginning the reverse transcription reaction at the end of the mRNA.

IV. Promoter Primers of the Invention

After the first strand cDNA is synthesized, a promoter primer is added to the 3′ end of the first strand cDNA. The promoter primer can be added with, for example, T4 RNA ligase or T4 DNA ligase. The promoter primer comprises gene regulatory sequence useful for driving expression of the cloned cDNA in vitro. Preferably, the promoter primer sequence comprises a promoter sequence recognized by the T7, T3 or SP6 RNA polymerase. An exemplary T7 consensus promoter sequence is TAATACGACTCACTATAGG. An exemplary T3 consensus promoter sequence is AATTAACCCTCACTAAAGG. An exemplary SP6 consensus promoter sequence is ATTTAGGTGACACTATAGA. However, any promoter sequence useful for in vivo or in vitro transcription can be used in the methods of the invention.

In circumstances where full length cDNA sequences are desired, the promoter primer acts to protect the 3′ end of the first strand cDNA molecule (corresponding to the 5′ end of the mRNA) from possible exonuclease degradation that can occur during the synthesis of the second cDNA strand. Addition of primers for the isolation of the 5′ ends of cDNAs have been described in, e.g., Schaefer, supra; Troutt, et al., Proc. Natl. Acad Sci. USA 89:9823-9825 (1992); and Apte, et al., BioTechniques 15:890-893 (1993). Once the promoter primer has been linked to the first strand, the cDNA molecules can be stored relatively stably.

The promoter primer can be comprised of a single or double stranded oligonucleotide. In some currently preferred embodiments, the promoter primer is a double stranded molecule with overhanging single stranded ends on each 3′ end. In one embodiment, one single stranded 3′ end is random and the other 3′ end is defined. The random end can therefore complement, and subsequently be ligated to, a pool of unknown cDNA molecules. One of skill in the art will recognize that the length of the random sequence will determine the efficiency of the ligation step. For example, the random overlap can have two to ten, and preferably four to six, base pairs. Double stranded primers can be ligated to the first strand cDNA with T4 DNA ligase.

Alternatively, a single stranded promoter primer is added to the 3′ end of the first strand cDNA. For instance, a single stranded oligonucleotide can be ligated to the 3′ end of the first strand cDNA with an enzyme such as T4 RNA ligase, which can ligate two single stranded molecules (see, e.g. Troutt et al., supra).

If desired, the promoter primer, as well as primer sequences incorporated into the 3′ end of the cDNA (i.e., a poly dT sequence), can be include restriction sites for cloning of the cDNA into a plasmid or other vector.

V. In vitro Transcription

The methods of the invention provide for the synthesis of large amounts of sense RNA using in vitro transcription reactions. By ligating the promoter primer to the 3′ end of the first strand cDNA molecule, the promoter is operably linked to direct transcription of sense strand RNA molecules in one simple step. Such constructs can be used to generate sense strand RNA in vivo or in vitro.

In vitro transcription involves providing all of the reagents necessary for transcription in a reaction mixture. Typically, the reactions comprise a template sequence (e.g., a cDNA sequence) operably linked to an RNA polymerase regulatory sequence, a RNA polymerase, and appropriate buffers and ribonucleotides. Kits for in vitro transcription reactions are available commercially from, e.g., Ambion, Inc.

An exemplary in vitro transcription reaction comprises, e.g., cDNA comprising a T7 promoter sequence in a volume of 101 μl, containing 2 μl each of ATP (75 mM), UTP, GTP and CTP, and 2 μl of 10×reaction buffer, and 2 μl of T7 enzyme mix. preferably, the reaction was incubated at 37° C. for 2-4 hours. After incubation, DNase I can be added to the reaction and incubated at 37° C. to digest the cDNA template. RNA can be extracted in phenol/chloroform and precipitated by addition of 0.1 volume of 3 M sodium acetate (pH 5.2), and 2.5 volume of cold ethanol and then dissolved in 40 μl of nuclease-free water.

As illustrated in the Figures, in some aspects of the invention, the RNA produced in the in vitro transcription reaction is isolated and reverse transcribed into cDNA molecules and then used as a template for in vitro transcription. By repeating the isolation and reverse transcription of RNA, small amounts of RNA from a cell can quickly and efficiently be amplified into large quantities.

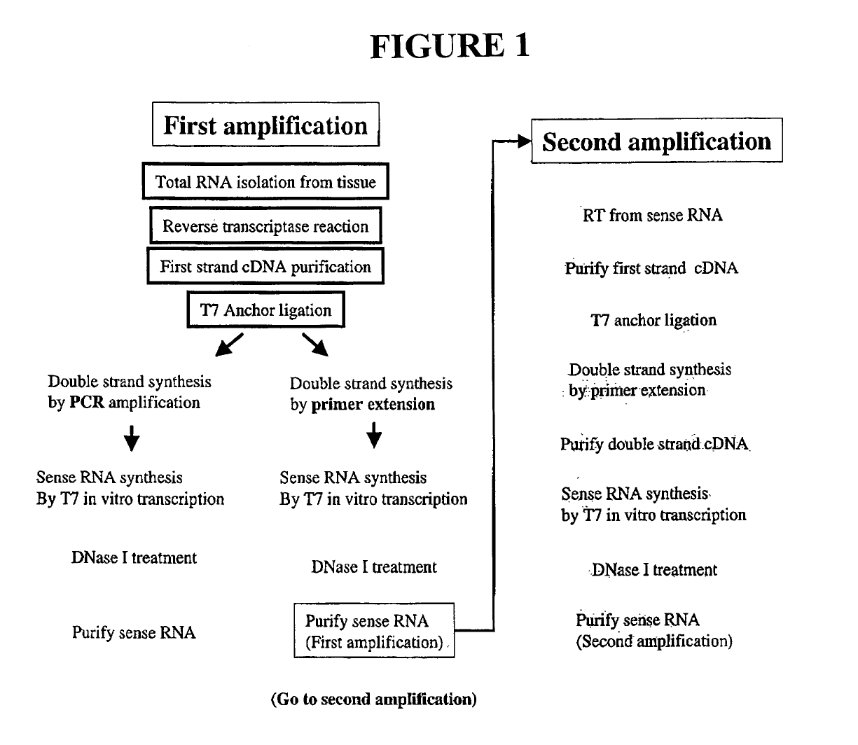

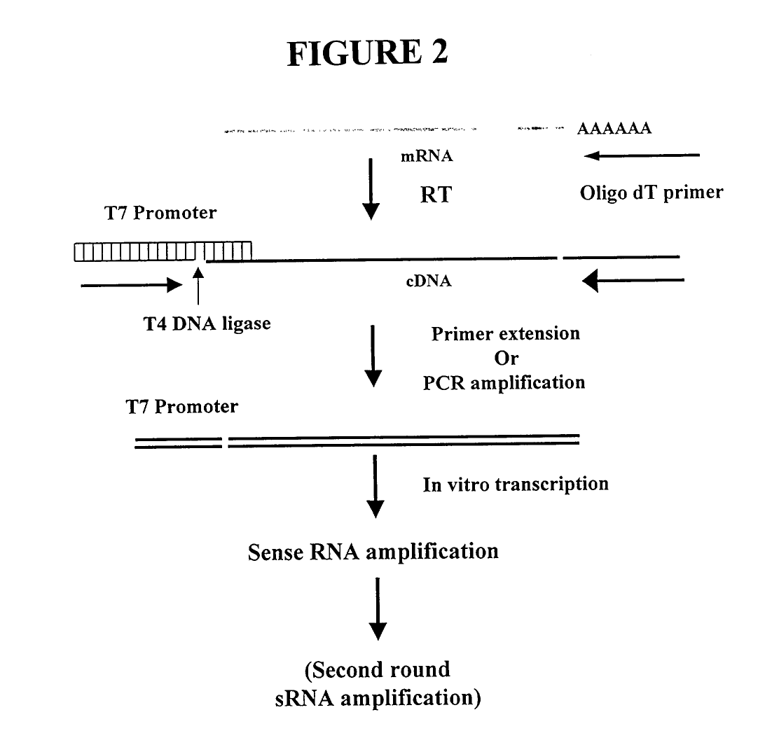

FIG. 2 displays one embodiment of the invention. The T7 promoter used in the Figures merely as an example of one promoter that can be used in the compositions and methods of the invention. In FIG. 1, messenger RNA is reverse transcribed into first strand cDNA using an oligo dT primer with a tailed adapter sequence. The first strand cDNA is then purified by RNase digestion or alkaline hydrolysis to remove RNA. The 3′ end of the first strand cDNA is ligated with a double stranded anchor adapter using T4 DNA ligase. The double stranded anchor adapter contains the T7 phage RNA polymerase promoter sequence. Because the adapter is added to the 3′ end of first strand cDNA, no sequence is lost at 5′ end mRNA, thereby generating full-length cDNAs. The T7 anchor sequence ligated to the cDNA ends serves a priming site for second strand cDNA synthesis. After ligation, the first strand cDNA can be converted into double strand cDNA by primer extension using a T7 promoter primer or by PCR amplification using a T7 promoter primer and a primer from oligo dT linker site. The double stranded cDNA products possess the T7 promoter upstream of all cDNA molecules. The double stranded cDNAs are then purified and used for amplification of sense RNA by in vitro transcription reactions. For PCR-amplified cDNA templates, one round of in vitro transcription is sufficient to generate 50-100 μg of sense RNA (e.g., producing up to 1000-fold amplification after 10 cycles of PCR). For primer extension of the cDNA templates comprising the T7 promoter, a second or more rounds of sense RNA is necessary if large amounts of RNA is desired. For example, first strand cDNA is synthesized from the sense RNA, the T7 promoter primer is ligated, second strand cDNA is synthesized and T7 in vitro transcription is performed

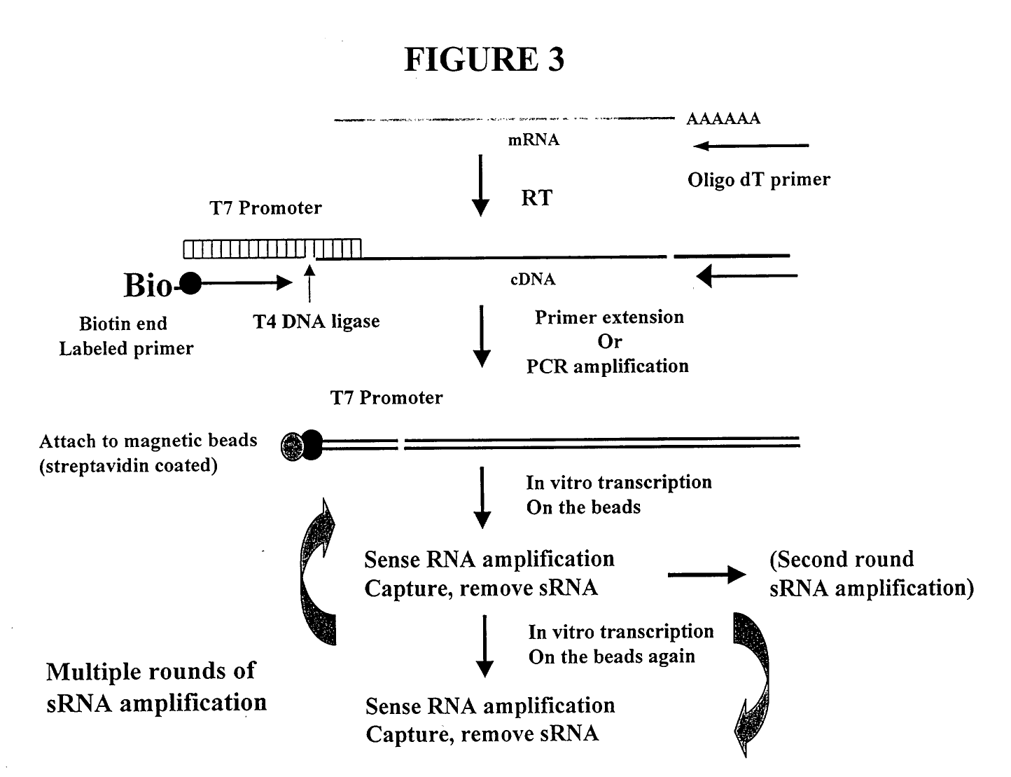

FIG. 3 illustrates a modified procedure where the amplification of sense RNA is performed on a solid support. In this embodiment, the method is basically similar to the method of FIG. 1, i.e., first strand cDNA is synthesized by a reverse transcriptase reaction and a T7 promoter primer is ligated to the 3′ ends of the first strand cDNA. However, in FIG. 2, a 5′ biotinylated primer that anneals to an anchored T7 promoter is used for primer extension or PCR amplification for second strand cDNA synthesis. Thus, double-stranded cDNAs have a biotin group incorporated which is attached to streptavidin-coated magnetic beads. In vitro transcription reaction is then carried out on the beads. After the reaction is completed, the magnetic beads are captured by a magnet and the supernatant that contains the newly amplified sense RNA is transferred to a fresh tube. Magnetic beads with the T7-anchored cDNAs can be resuspended with a fresh T7 RNA polymerase cocktail for sense RNA amplification in vitro again. This process can be repeated for multiple rounds and sense RNA can be collected each time.

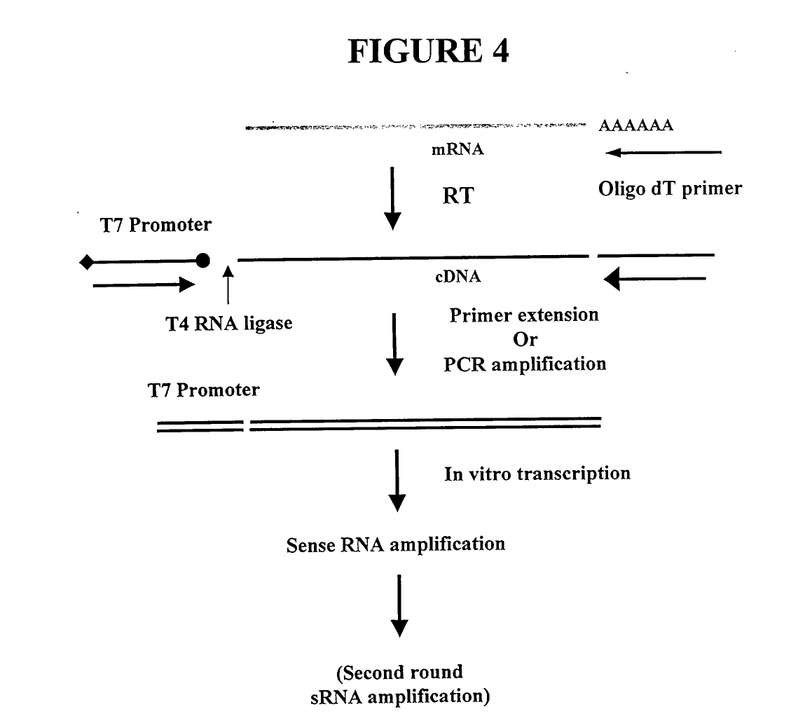

FIG. 4 illustrates a method for adding a single-stranded T7 promoter anchor sequence to 3′ ends of first strand cDNA using T4 RNA ligase. In this embodiment, a RNA ligase, such as T4 RNA ligase, is used to catalyze the ligation between two single stranded nucleic acids. The T7 promoter anchor sequence ligated to the 3′ ends of cDNAs provides a hybridization site for a primer used for second strand cDNA synthesis. The basic procedures are the same as FIG. 1. Difference in FIG. 3 include that the oligo dT primer for a reverse transcriptase reaction is blocked at the 5′ end with amine group that prevents the cDNA ends from self ligation. In this embodiment, the T7 promoter primer is a single-stranded oligo nucleotide and its 5′ end is phosphorylated to allow ligation with the 3′ end OH group from first strand cDNA. After the T7 promoter anchor sequence is ligated to the 3′ ends of cDNA, second strand cDNA is synthesized by primer extension or PCR amplification as described above. Sense RNA (sRNA) amplification is also carried out as described above.

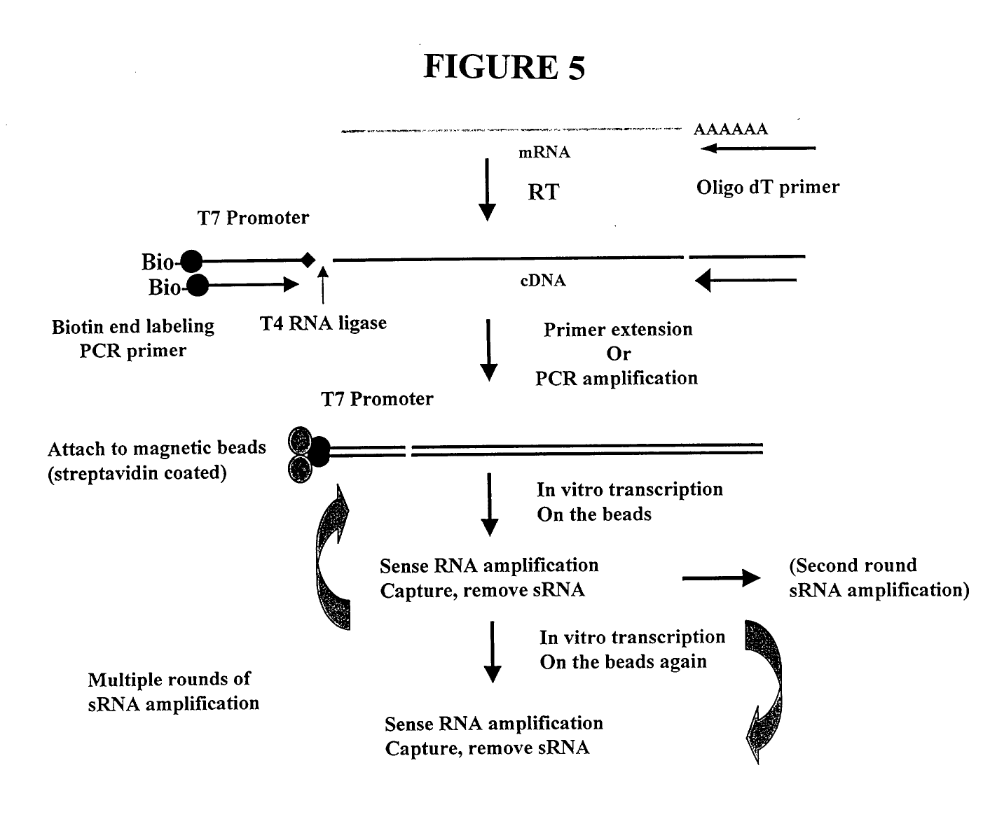

FIG. 5 illustrates a variation of the method depicted in FIG. 2 that employs T4 RNA ligase to ligate a T7 promoter primer to the 3′ ends of the first strand cDNA. A biotinylated primer that binds to T7 anchor is used for primer extension or PCR amplification for second strand cDNA synthesis. All other procedures are the same as in FIGS. 2-3.

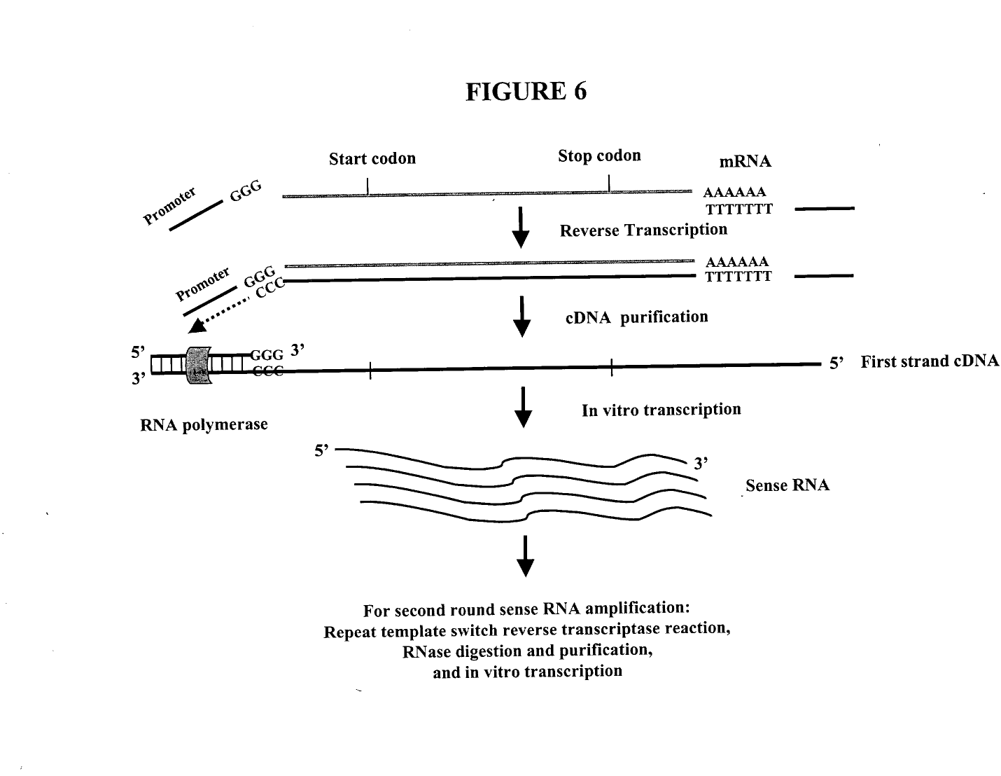

FIG. 6 illustrates a strategy for sense mRNA amplification via a template switching mechanism using a double-stranded promoter and a single-stranded template. In this method, like the method of FIG. 1, messenger RNA is reverse transcribed into first strand cDNA using an oligo dT or a random primer. However, here, a double-stranded promoter sequence is incorporated by a “template switch” mechanism. A single stranded primer containing a promoter (for example, a T7, a T3 or a SP6 promoter) sequence is synthesized with a 3′ nucleotide overhang of about 3, 5, 7 or 10 nucleotides, for example --promoter-GGG-3′--. The promoter primer sequence with the 3′ overhang is added to a cDNA synthesis reaction in the presence of an oligo dT or random primer and a reverse transcriptase. During the reverse transcriptase reaction, a complementary overhang nucleotide sequence, for example CCC, is added to the reaction and incorporated into 3′ end of the first strand of cDNA by reverse transcriptase. The promoter-nucleotide overhang-3′ primer will anneal to the complementary overhang nucleotide sequence. The reverse transcriptase will then switch templates from the messenger RNA to the promoter-nucleotide overhang-3′ primer, extending the first strand cDNA sequence to make a double-stranded promoter sequence. Once cDNA synthesis is complete, two approaches can be used to generate sense strand mRNA from the mRNA/cDNA heteroduplex.

In a first approach, the the mRNA strand annealed in the mRNA/cDNA heteroduplex can be removed by RNase digestion, for instance, by using RNase H. This treatment leaves the promoter region still double-stranded while the cDNA is single-stranded. The product is then purified and subjected to in vitro transcriptase reaction to generate sense RNA.

In a second approach, the mRNA/cDNA heteroduplex is subjected to an RNase (for example, RNase H), a DNA polymerase and a DNA ligase. The RNase nicks the mRNA along the heteroduplex while the DNA polymerase catalyzes nick translation for second strand cDNA synthesis, and the DNA ligase ligates the nicks. This approach produces double-stranded cDNA with a double stranded promoter sequence incorporated at the 3′ end of the antisense cDNA strand, which allows synthesis of sense RNA by an in vitro transcription reaction.

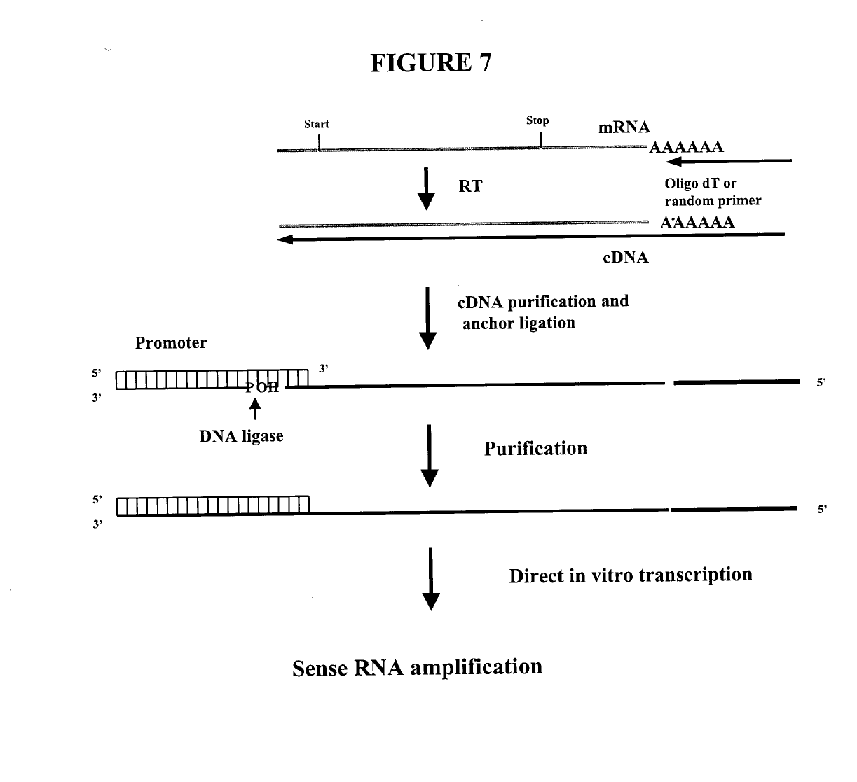

FIG. 7 illustrates a strategy for sense mRNA amplification via in vitro transcription using a double-stranded promoter and a single-stranded template. In this method, as with the “switch template” method, sense strand mRNA is generated from a single-strand cDNA template sequence driven by a double-stranded promoter sequence. Like the method of FIG. 1, messenger RNA isolated from a biological sample is reverse transcribed into first strand cDNA using an oligo dT or a random primer. A double-stranded adaptor containing RNA polymerase promoter sequences is then added in a ligase reaction to the 3′ ends of the transcribed cDNA molecules. The final product generated by this procedure is a single-stranded cDNA template with a double-stranded promoter sequence at the 3′ end. This product can be directly used for in vitro transcription reaction to generate sense strand mRNA in large quantities.

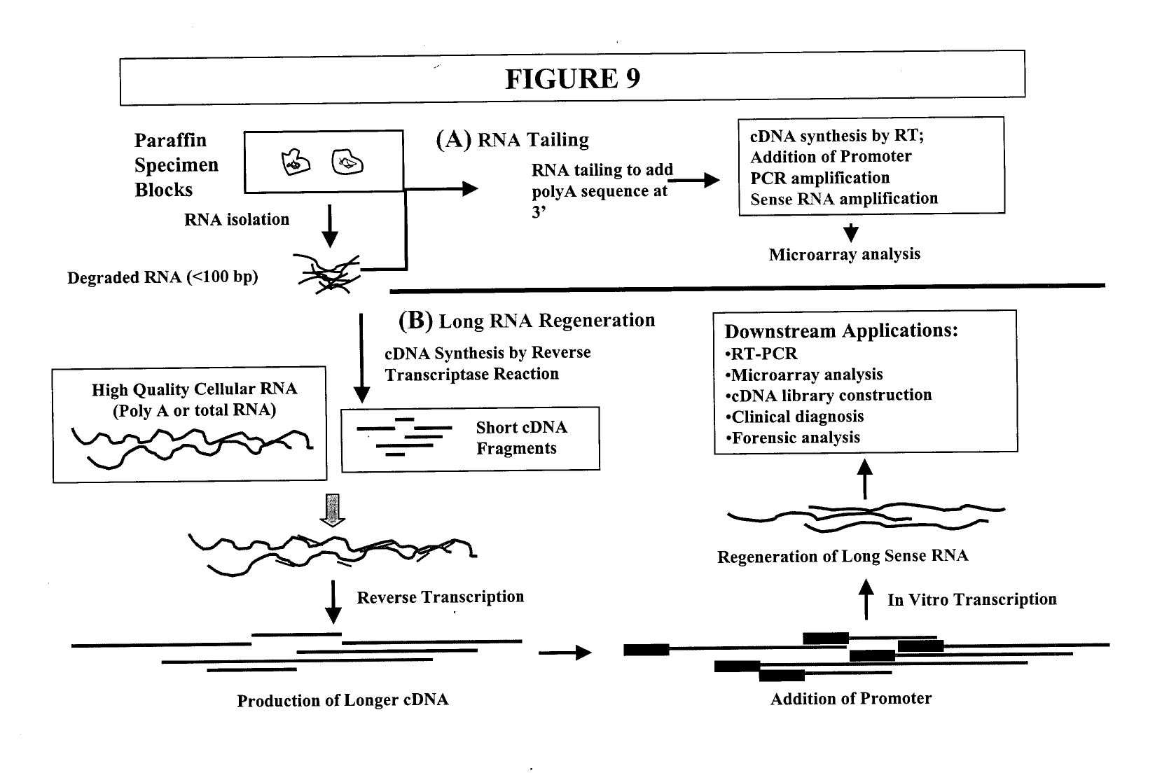

FIG. 9 illustrates gene expression profiling by microarray analysis using RNA samples isolated from paraffin blocks. Two approaches for sense RNA sequences are provided, “mRNA tailing” and “long mRNA regeneration.”

In the “RNA tailing” approach, partially degraded mRNA is isolated from a biological sample, such as a paraffin block containing tissue, and a poly A sequence is ligated to the 3′ end of the isolated RNA sequences using terminal transferase. A first strand of cDNA is synthesized by reverse transcription reaction and a RNA promoter primer sequence is ligated to the 3′ end of the first strand of cDNA. A second strand of cDNA that is complementary to the first strand of cDNA is synthesized, either by PCR amplication or primer extension. Sense RNA is then amplified by an in vitro transcriptase reaction.

In the “long RNA regeneration” approach, partially degraded RNA is isolated from a biological sample, such as a paraffin block containing a first tissue, and cDNA sequences complementary to the partially degraded RNA are synthesized by a reverse transcription reaction. Full length mRNA sequences are then isolated from cells of a second tissue that is the same tissue type as the first tissue. The partial length cDNA sequences are hybridized to the full length mRNA sequences isolated from the second tissue. cDNA corresponding to full length mRNA messages is synthesized by an extension reaction using reverse transcriptase. A RNA promoter primer is then ligated to the 3′ end of the full length cDNA sequences. Sense RNA is amplified using an in vitro transcription reaction.

The sense RNA generated using either approach can be synthesized using labeled nucleotides, for instance with nucleotides labeled with a fluorescent dye, for example, cy3 or cy5. The labeled amplified sense RNA sequences can be directly hybridized to a microassay slide, for instance one comprising a cDNA microarray. Sense RNA generated using the “RNA tailing” approach finds use in RT-PCR analysis, such as Taqman and as probes in nucleic acid hybridization. Sense RNA produces using the “long RNA regeneration” approach finds particular use in RT-PCR (Taqman) for quantitation, as probes in nucleic acid hybridization, in library construction, for forensic analysis and in clinical diagnosis.

As described above, the methods of the invention are useful to amplify large amounts (micrograms) of long sense-strand RNA. The ability to amplify sense strand RNA is of particular utility for constructing expression (e.g., cDNA) libraries when very small amounts of initial RNA is available (e.g, from small amounts of tissue or from single cells).

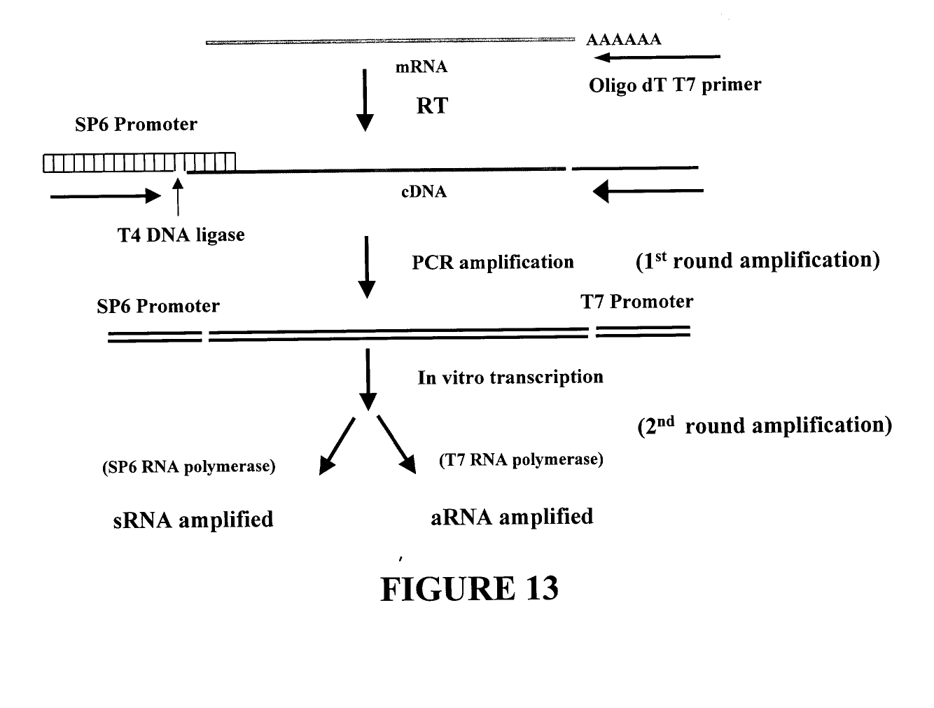

The invention also provides for a method of generating long antisense strand RNA. As illustrated in FIG. 13, first strand cDNA is synthesized using an oligo dT-first promoter primer (here, T7). An upstream adaptor primer comprised of a second promoter (here, SP6) having a 3′ overhanging single stranded sequence is incorporated onto the 3′ end of the first strand cDNA. PCR is used to amplify double-stranded cDNA templates. Long sense strand RNA is generated when the RNA polymerase for the second promoter is used, while long antisense strand RNA is generated when the RNA polymerase for the first promoter is used.

VI. Uses of Sense RNA Produced by the Methods of the Invention

Those of skill in the art will recognize that sense RNA and amplification of sense RNA has numerous uses for biological research and commercial products. For instance the amplified sense RNA of the invention can be used for any purpose mRNA is typically used for. Of course, the amplified RNA of the invention is particularly useful because it can be obtained in large quantities.

The sense RNA can be used as a hyridization probe for any experiment where RNA probes are useful. Moreover, the RNA can be used in differential display experiments. Alternatively, the sense RNA is useful for 5′ exon capture experiments.

Two examples of use of amplified sense RNA are described in more detail below.

a. Microarray Hybridization

Amplified sense RNA can be used directly as probes for hybridization experiments. For example, sense RNA can be used directly as probes for hybridization of nucleic acid microarrays (e.g., gene chips). Microarray technology is well known and is described in e.g., Lockhart et al. Nature 405:827-836 (2000). Alternatively, sense RNA can be used as a template to generate labeled single-stranded cDNA probes using reverse transcriptase.

In some embodiments, the invention provides methods of synthesizing sense RNA for use as probes of nucleic acid microarrays. Sense RNA probes are useful for probing any nucleic acid microarray that comprises complementary polynucleotides. Such arrays include double stranded or single-stranded polynucleotide microarrays.

Labeled nucleotides can therefore be introduced into the synthesized RNA without additional steps. Examples of labeled nucleotides include fluorescent nucleotides (e.g., Cy3 or Cy5 conjugated to nucleotides such as dUTP, available from e.g., Amersham) and radioactively-labeled nucleotides. The labeled nucleotides are introduced into the sense RNA by supplying labeled nucleotides in sufficient concentrations into the in vitro transcription reaction to produced labeled sense RNAs.

Alternatively, in some aspects, sense RNA generated by the methods of the invention can be used as a template for a reverse transcription reaction to generate labeled single-stranded cDNA. In these embodiment, sense strand RNA is reverse transcribed using standard procedures in the presence of labeled nucleotides as described above. The labeled single-stranded cDNAs are useful as probes of double and single stranded mmicro arrays, including oligo-based microarrays (e.g., from Affymetrix Inc.)

b. cDNA Normalization/Hybridization

The present invention also provides methods of using sense RNA as a driver sequence in library normalization or other subtractive methods. The construction of normalized expression libraries using subtractive hybridization techniques have been described previously. See, e.g., Soares et al., Proc. Natl. Acad. Sci. USA 91:9228-9232 (1994); Bonaldo et al., Genome Res. 6:791-806 (1996); Caminci et al., Genome Res. 10: 1617-1630 (2000). Driver polynucleotides are generally used to remove unwanted sequences from an expression library. For example, a normalized library can be constructed by subtracting (i.e., removing) cDNAs expressed in a healthy tissue from cDNAs expressed in diseased tissue. Similarly, in normalization methods, high copy number sequences are removed from a sample. The resulting normalized library will be highly enriched for disease-specific cDNAs.

Thus, the present invention is useful where it is desired to have driver RNA from a particular tissue. For example, RNA can be isolated from the tissue, amplified according to the methods of the present invention, and sense RNA can be synthesized and biotinylated. The biotinylated driver RNA is then hybridized to cDNA from another library, and then the biotinylated driver RNA is removed, thereby leaving a subtracted library. Those of skill in the art, however, will recognize that there are numerous ways to construct subtractive libraries using the sense RNA produced according to the methods of the invention.

VII. Kits: Use in Diagnostic, Research, and Therapeutic Applications

For use in diagnostic, research, and therapeutic applications disclosed here, kits are also provided by the invention. In the diagnostic and research applications such kits can include any or all of the following: assay reagents, buffers, specific nucleic acids or antibodies, hybridization probes and/or primers, and the like. A therapeutic product may include sterile saline or another pharmaceutically acceptable emulsion and suspension base.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for generating long sense or antisense strand RNA. Such kits can be prepared from readily available materials and reagents. For example, such kits can comprise one or more of the following materials: an upstream promoter primer comprising a 3′ overhanging single stranded sequence, wherein the promoter is a T7, T3, or SP6 promoter, wherein the 3′ overhanging sequence can be comprised of 6-10 random nucleotides; a downstream promoter comprising an oligo dT sequences or a sequence of random nucleotides, enzymes for carrying out the reactions of the method, including DNA and RNA polymerases, DNA and RNA ligases, a reverse transcriptase, buffers for carrying out the reactions, reaction tubes, and instructions for generating long sense or antisense strand RNA.

A wide variety of kits and components can be prepared according to the present invention, depending upon the intended user of the kit and the particular needs of the user. Diagnosis would typically involve evaluation of a plurality of expressed genes, usually from a biological sample. The presence or absence of genes in a population pool of RNA that has been amplified into long sense or antisense strand RNA can be evaluated using microarray technologies, described above.

### EXAMPLES

### Example 1

This Example illustrates the creation of a cDNA construct for production of sense RNA.

RNA Isolation

Total RNA was isolated from cell cultures or from tissue samples, or from microdissected samples with laser capture microscopy using standard procedures such as Trizol reagents (Life Technologies). Total RNA was treated with RNase-free DNase to remove DNA contamination. Total RNA was ethanol precipitated and dissolved in nuclease-free water. Poly (A) RNA was isolated from total RNA using oligo (dT) cellulose chromatography or using Oligotex mRNA isolation kit (Qiagen).

First Strand cDNA Synthesis

The reverse transcriptase reaction was carried out in small reaction volume (5 μl). Briefly, 1 μl of RNA was mixed with 4 μl of master mixture solution to bring the volume to 5 μl containing 50 mM Tris-HCl, pH 8.3, 75 mM KCl, 3 mM MaCl₂, 10 mM dithiothreitol, 0.6 mM dNTPs, 4 units of RNase inhibitor (Promega), 0.4 μM oligo dT₁₈ linker primer, and 40 units of SuperScript II reverse transcriptase (Life Technologies, Inc.). The mixture was briefly centrifuged and incubated in a MicroIncubator M-36 (Taitec, Inc.) at 42° C. for 60 minutes, and 50° C. for 30 minutes, followed by incubating at 65° C. for 10 minutes in a water bath to inactivate the enzyme.

Purification of First Strand cDNA

After the reverse transcriptase reaction, RNA was removed by treatment with RNase. For RNase treatment, the cDNA was heated at 92° C. for 2 minutes and immediately chilled on ice for 2 minutes, and briefly centrifuged. One microliter of mixture containing 0.1 unit of RNase I (Promega) and 0.2 units of RNase H (Life Technologies, Inc.) was added to the cDNA. The mix was incubated for 20 minutes at 37° C. for RNA digestion. After that, 2.5 volume of cold ethanol (15 μl) was added and mixed by vortex. After incubating at −80° C. or on dry ice for 5 minutes, the cDNA was precipitated by centrifuge at 16,000 g for 15 minutes at 4° C. The pellet was then rinsed with 70% cold ethanol, air dried and redissolved in distilled water. The cDNA was further purified using Microcon-30 centrifugal filter device (Millipore) to remove access primers, and salt according to the product instruction. The cDNA was eluted from the Microcon-30 filter and was used for T7 promoter anchor ligation.

T7 Promoter Primer Ligation

The T7 promoter primers were designed as follows:

For double-stranded primer ligation using T4 DNA ligase, the following two complementary primers were used to annealed together. The primers contain T7 RNA polymerase promoter sequence (underlined):

T7N6 (+): 5′-NH2-CGGCCAGTGAATTGTAATACGACTCACTATAGG

CGCNNNNNN-NH₂-3′

T7-P-N (−): 5′-P-GCGCCTATAGTGAGTCGTATTACAATTCACTGG

CCGTCGTTT-NH₂-3′.

The primer T7N6 contained degenerate sequences at 3′ end. Both ends of the T7N6 primer was blocked by an amine group during primer synthesis. The primer T7-P-N contained a phosphate group at the 5′ end for ligation to occur and was blocked with an amine group at the 3′ end. All primers were purified using 20% denaturing acrylamide gel electrophoresis using standard procedure.

The primers T7N6 and T7-P-N (at 50 μM) were annealed in a 0.6-ml tube in 20 μl of 1×ligation buffer solution (50 mM Tris-HCl, 10 mM MgCl₂, 1 mM ATP, 10 mM dithiothreitol and 25 μg/ml bovine serum albumin) by heating at 92° C. for 2 minutes in a heating block, and slowly cooled down to room temperature by tuning off the heating block. After annealing, the adapter was further diluted with 1×ligation buffer solution into 10 μM, and stored at −20° C. in aliquots prior to use.

Ligation of T7 Promoter Sequence to 3′cDNA Ends Using T4 RNA Ligase.

A single-stranded T7 promoter anchor primer (T7-P-N) was used for ligating to 3′ ends of first strand cDNA using T4 RNA ligase. The primer T7-P-N contained a phosphate group at 5′ end for ligation to occur and was blocked with an amine group at 3′ end.

T7-P-N (−): 5′-P-GCGCCTATAGTGAGTCGTATTACAATTCACTGGCCGTCGTTT-NH₂-3′

First strand cDNA was purified and dissolved in distilled water. The cDNA was ligated to the anchor primer T7-P-N in 20 μl of volume containing the first strand cDNA, 20 pmoles of anchor primer, 50 mM Tris-HCl (pH 8.0), 10 mM MgCl₂, 1 mM hexamine cobalt chloride, 20 μM ATP, 25% (w/v) PEG 8000, 10 μg/mL of bovine serum albumin and 10 units of T4 RNA ligase (New England Biolabs, Beverly, Mass.) at 22° C. for 12-16 hours. The ligation reaction was extracted with phenol/chloroform, and purified using Microcon-30 centrifugal filter device (Millipore) to remove access anchor primers, and salt according to the product instruction. The anchor ligated cDNA was eluted from the Microcon-30 filter and used for second strand full-length cDNA synthesis with primer extension or by PCR amplification.

Adapter Ligation to 3′ cDNA Ends Using T4 DNA Ligase

T4 DNA ligase catalyzes ligation of double stranded DNA. The double stranded T7 promoter primer used in this example contained 6 random base pairs at the 3′ end that annealed with the 3′ end cDNA sequence to form a partial double strand region for the ligation to occur between a phosphate group (PO₄) on the adaptor and a hydroxyl group (OH) on 3′ end of cDNA. The double-stranded promoter primer was ligated to the first strand cDNA ends.

A T4 DNA ligase reaction was carried out 10 μl volume containing the purified first strand cDNA, 1 μl of 10×T4 DNA ligase buffer (New England Biolabs), 1 μl (20 pmoles) of double stranded T7 anchor (T7N6/T7-P-N), and 500 units of T4 DNA ligase (2000 units/μl, New England Biolabs). The ligation reaction was incubated for 16 hours at 14° C. The reaction was then heated at 65° C. for 10 minutes to inactivate the enzyme. After anchor ligation, the reaction was further purified by phenol/chloroform extraction, Microcon 30 centrifugal filter unit, and eluted in distilled water as above, and ready for second strand cDNA synthesis using primer extension or PCR amplification.

### Example 2

This example illustrates second strand synthesis by primer extension.

Primers used for primer extension or for PCR amplification include the following:

T7PR-2:5′-AAACGACGGCCAGTGAATTGTAATACGACTCACTATAGG-3′

This primer is T7PR-2, which is complementary to the primer T7-P-N, was used for primer extension or for PCR amplification experiments described below to convert single-stranded first strand cDNA into double stranded cDNA.

Another primer T7PR-2-Bio was also designed for primer extension or for PCR amplification to convert single-stranded first strand cDNA into double stranded cDNA. The primer sequence of T7PR-2-Bio was the same as T7PR-2 but contained a 5′ biotin group. This primer resulted in double-stranded cDNA with a 5′ end biotin labeling after primer extension or PCR amplification.

T7PR-2-Bio: 5′-Bio-AAACGACGGCCAGTGAATTGTAATACGACTCACTATAGG-3′.

Primer extension was carried out for second strand cDNA synthesis. Briefly, T7 promoter anchor-ligated first strand cDNA was mixed with 20 pmoles of primer T7-PR-2, 0.2 mM each of dNTPs, 1×high salt buffer, and 2.5 units of Taq Plus Long enzyme (Stratagene) in 50 μl of volume. The reaction was initially denatured at 94° C. for 2 minutes, followed by 10 cycles of 68° C. for 1 minutes and 72° C. for 5 minutes. The products were extracted with phenol/chloroform, and purified using Microcon-30 centrifugal filter device (Millipore) to remove access primers, and salt.

### Example 3

This example illustrates second strand synthesis using PCR.

Second strand cDNA was synthesized and amplified by the polymerase chain reaction. To avoid sample skewing caused by over-amplification, PCR was performed with a limited number of cycles. Takara LA Taq polymerase mix (Tara Shuzu Co., Ltd, PanVera, Madison, Wis.) was used for PCR amplification. PCR was carried out in 50-100 μl of volume using one fifth volume of the purified ligation anchored first strand cDNA, 40 pmoles of upstream (T7 promoter) and downstream (oligo dT linker) primers, 200 μM dNTPs, and 1 μl of Takara LA Taq polymerase mix according to the manufacturer's instruction. PCR was performed for 10-15 cycles as follows: 94° C. for 1 minute for initial denaturation, 10-15 cycles of 98° C. for 10 seconds and 68° C. for 6 minutes, followed by final extension at 68° C. for 10 minutes. After PCR amplification, one-tenth of PCR product was analyzed on a 1% agarose gel in IX TAE buffer stained with ethidium bromide. The remaining PCR products were digested with 1 μl of protainase K (Roche) at 50° C. for 15 minutes and extracted with phenol/chloroform. The amplified PCR products were further purified three times using Microcon 100 centrifugal filter units (Millipore) to remove primers, primer dimers, and salts. After elution from the filter units with water, the cDNA was quantified using UV spectrophotometer at A260.

### Example 4

This example illustrates the purification of double-stranded cDNA.

In vitro transcription was carried out using the Megascript kit (Ambion) as follows. T7 promoter-cDNA was incubated in 20 μl of volume containing 2 μl each of ATP (75 mM), UTP, GTP and CTP, and 2 μl of 10×reaction buffer, and 2 μl of T7 enzyme mix. The reaction was incubated at 37° C. for 2-4 hours. After incubation, 1 μl of DNase I (2 units/μl) was added to the reaction and incubated at 37° C. for 20 min to digest the cDNA template. RNA was extracted once with phenol/chloroform, and once with chloroform and precipitated by addition of 0.1 volume of 3 M sodium acetate (pH 5.2), and 2.5 volume of cold ethanol. The pellet was dissolved in 40 μl of nuclease-free water. The RNA solution was further purified by a spun column (NucAway Spin Column; Ambion) to remove unincorporated nucleotides. The RNA quantity was measured by UV-spectrophotometer. An aliquot of sense RNA was analyzed on 1% agarose gel stained with ethidium bromide.

### Example 5

This example shows a comparison of the present method for sense strand mRNA amplification with commercially available methods for antisense mRNA amplification.

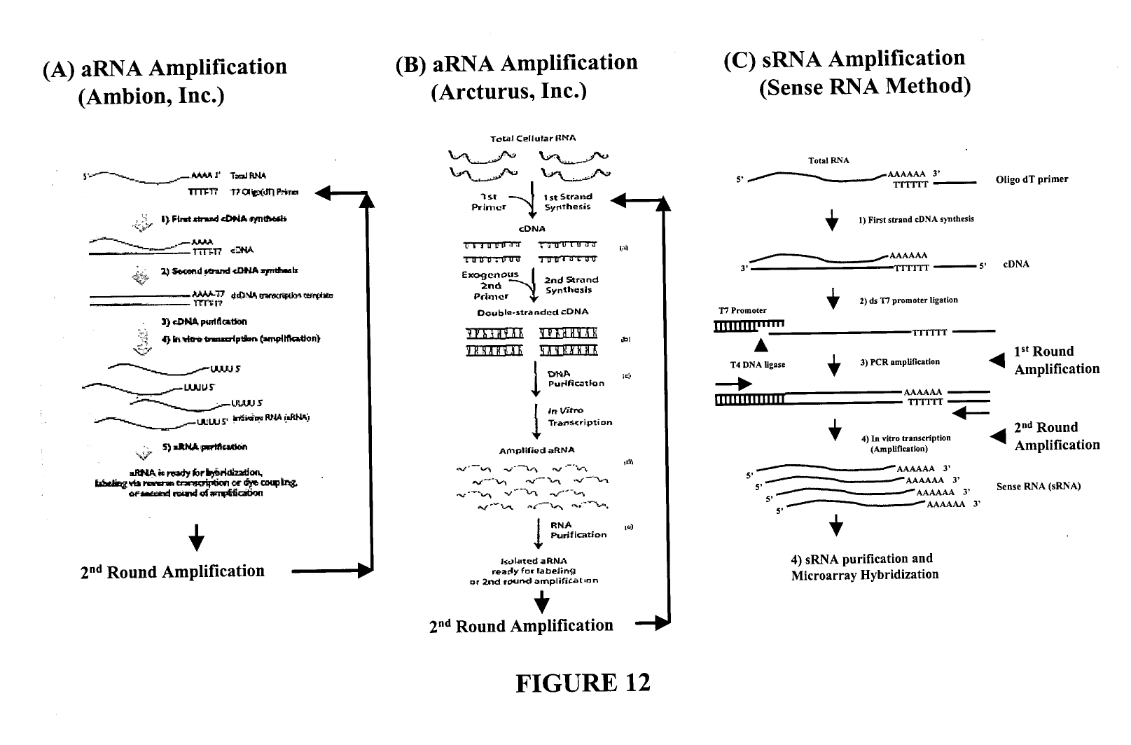

FIG. 12 illustrates a comparison of methods for in vitro RNA amplification. One approach of the presently described methods of amplification of sense strand mRNA was compared with commercially available methods for amplification of antisense RNA that can be carried out using kits purchased from, for example, Ambion, Inc. or Arcturus, Inc. First round aRNA amplification using the kit from Ambion, Inc. normally takes about 1 day (about 27-28 hours). First round aRNA amplification using the kit from Arcturus, Inc. takes about 8 hours. For first and second round amplification combined, it takes at least about 2-3 days using either commercial kit. By comparison, amplification of sense strand mRNA following the illustrated protocol requires only about 8 hours to complete both the first and second rounds of amplification: RT (about 1.5 hours), promoter primer adapter ligation (about 1.5 hours), PCR amplification (about 15 cycles; about 2.5 hours) and in vitro transcription for sRNA amplification (about 2.5 hours).

Comparison of the resulting sense and antisense mRNA from the three procedures demonstrated that amplification of sense mRNA by ligating a promoter primer to the 3′ end of the first cDNA strand generates a mRNA population pool with significantly longer sequences than either of the commercial antisense mRNA amplification methods. In a gel comparison of the mRNA produced by all three methods, the longest antisense strand mRNA sequences produced by either the Ambion or Arcturus methods after a first round of amplification was about 1.5 kb; the longest mRNA sequences after the second round of amplification was about 0.6 kb. By contrast, the longest sense strand mRNA sequences after the second round of amplification using the illustrated method of the present invention was about 2.2 kb. Therefore, the present method for sense strand mRNA amplification produced mRNA sequences that were about 50% longer than first round amplified antisense RNA, and about 250% percent longer than second round amplified aRNA using commercial kits.

Validation of long sense strand mRNA molecules was confirmed by amplifying using RT-PCR a 5′ coding region of clathrin that is about 6 kb from the 3′ end. From all sense strand mRNA samples tested, this 5′ coding region that is about 6 kb upstream from the 3′ end was amplified. From antisense RNA samples prepared using commercial kits that employ a promoter primer ligated to the 5′ end of the first strand of cDNA, this 5′ clathrin coding region did not amplify.

Therefore, the present methods for sense strand mRNA amplification generate longer sequences of mRNA than commercially available technologies. The ability to efficiently generate full length coding sequences will facilitate more accurate and representative expression profiling of an mRNA population pool.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method of generating a long sense strand of RNA, the method comprising,
providing a first strand of cDNA comprising a 5′ and a 3′ end;
incorporating a promoter primer comprising a promoter regulatory element onto the 3′ of the first strand cDNA; and
initiating transcription from the cDNA, thereby generating a long sense strand RNA.

2. The method of claim 1, wherein the promoter primer is double-stranded.

3. The method of claim 2, wherein incorporating the promoter primer is ligating said promoter primer to the first strand cDNA by T4 DNA ligase.

4. The method of claim 2, further comprising the step of synthesizing a second strand cDNA complementary to the first strand cDNA before initiating transcription from the cDNA.

5. The method of claim 1, wherein the promoter primer is single stranded, and further comprising an additional step of synthesizing a second strand cDNA complementary to the first strand cDNA, thereby incorporating the promoter primer into a double-stranded cDNA, is carried out before initiating transcription of the cDNA.

6. The method of claim 1, wherein the method further comprises PCR amplification of the double stranded cDNA.

7. The method of claim 1, wherein the promoter regulatory element is from a promoter selected from the group consisting of the T7, T3 an SP6 promoter.

8. The method of claim 1, wherein the primer is biotin-labeled.

9. The method of claim 5, wherein the double-stranded cDNA is purified with magnetic beads.

10. The method of claim 9, wherein the transcription of the cDNA occurs when the cDNA is anchored to the magnetic beads.

11. The method of claim 9, wherein the magnetic beads are linked to streptavidin.

12. The method of claim 1, wherein the first strand cDNA comprises a poly dT sequence.

13. The method of claim 5, wherein incorporating the promoter primer is ligating said promoter primer to the first strand cDNA by T4 RNA ligase.

14. The method of claim 5, wherein the single stranded promoter primer is phosphorylated on the 5′ end.

15. The method of claim 2, wherein the promoter primer comprises an overhanging single stranded sequence at least partially complementary to the first strand cDNA.

16. The method of claim 2, wherein the promoter primer comprises random nucleotides on the 3′ end of the primer.

17. The method of claim 16, wherein the promoter primer comprises 6-10 random nucleotides on the 3′ end of the primer.

18. The method of claim 1, wherein the transcription comprises incorporation of labeled nucleotides into the sense strand mRNA, thereby synthesizing a labeled sense strand mRNA.

19. The method of claim 18, wherein the labeled nucleotides are fluorescent nucleotides.

20. The method of claim 18, wherein the method further comprises probing a polynucleotide array with the labeled sense strand mRNA.

21. The method of claim 1, further comprising reverse transcribing the sense strand RNA, thereby synthesizing a single-stranded cDNA probe.

22. The method of claim 21, wherein the reverse transcription step is performed in the presence of labeled nucleotides, thereby synthesizing a labeled single-stranded cDNA probe.

23. The method of claim 22, wherein the nucleotides are labeled with fluorescent dye.

24. The method of claim 23, wherein the fluorescent dye is selected from the group consisting of cy3 and cy5.

25. The method of claim 1, wherein said method further comprises the step of isolating mRNA from a biological sample.

26. The method of claim 25, wherein said biological sample comprises a submicrogram quantity of total RNA.

27. The method of claim 26, wherein said biological sample comprises partially degraded mRNA.

28. The method of claim 27, wherein said biological sample is from paraffin-embedded tissue.

29. A method of generating a mixture of sense strand of mRNAs, the method comprising,
providing a pool of mRNA from a biological sample;
synthesizing a pool of first strand cDNAs comprising a 5′ and a 3′ end using the pool of mRNA isolated from a biological sample as a template;
incorporating a promoter primer comprising a T7, T3 or SP6 promoter onto the 3′ of the first strand cDNAs; and
initiating transcription of the double-stranded cDNAs, thereby generating a mixture of sense strand mRNAs.

30. The method of claim 29, wherein the promoter primer is double-stranded.

31. The method of claim 30, further comprising the step of synthesizing a second strand cDNA complementary to the first strand cDNA before initiating transcription of the cDNA.

32. The method of claim 29, wherein the promoter primer is single stranded, and wherein the additional step of synthesizing a second strand cDNA complementary to the first strand cDNA, thereby incorporating the promoter primer into a double-stranded cDNA, is carried out before initiating transcription of the cDNA.

33. The method of claim 29, wherein the method further comprises normalizing a cDNA library with the mixture of sense strand mRNAs.

34. The method of claim 33, wherein the mixture of sense strand mRNAs are biotinylated and the method further comprises the steps of
contacting in a solution the mixture of sense strand mRNAs with the cDNA library, thereby forming RNA/DNA hybrids; and
separating the hybrids from solution.

35. A method of generating a long antisense strand of RNA, the method comprising,
synthesizing a first strand of cDNA comprising a 5′ and a 3′ end using an oligo dT-first promoter primer comprising a first promoter regulatory element;
incorporating a promoter primer comprising a second promoter regulatory element onto the 3′ of the first strand cDNA;
synthesizing a second strand cDNA complementary to the first strand cDNA, thereby incorporating the second promoter primer into a double-stranded cDNA and
initiating transcription of the cDNA from the first promoter primer, thereby generating a long antisense strand RNA.

36. A kit, comprising a double-stranded promoter primer comprising a 3′ overhanging single stranded sequence.
